(19) United States
(12) Patent Application Publication
(10) Pub. No.: US 2021/0106849 A1
(43) Pub. Date: Apr. 15, 2021

(54) Method and Kit for Treating Brain Tumor by using an Ultrasound System

(71) Applicant: NaviFUS Co. Ltd., Taipei City (TW)

(72) Inventors: Hao-Li LIU, Taoyuan City (TW); Po-Chun CHU, Taipei City (TW); Ting-Kuang CHANG, Taipei City (TW); Kuo-Chen WEI, Taoyuan (TW); Pin-Yuan CHEN, Taoyuan (TW); Chiung-Yin HUANG, Taoyuan (TW)

(73) Assignee: NaviFUS Co. Ltd., Taipei City (TW)

(21) Appl. No.: 15/975,672

(22) PCT Filed: Nov. 10, 2016
(86) PCT No.: PCT/CN2016/105293

§ 371 Date: May 9, 2018

Related U.S. Application Data
(60) Provisional application No. 62/253,805, filed on Nov. 11, 2015.

(51) Int. Cl. A61N 7/00; C07K 16/22; A61P 35/04; A61K 51/10

(52) U.S. Cl. CPC A61N 7/00; C07K 16/22; A61P 35/04; A61K 9/0019; A61N 2007/0021; A61N 2007/0039; A61K 51/1045

(57) ABSTRACT

The present invention relates to a method for reducing an effective amount of Bevacizumab required for treating brain tumor. The present invention also provides a kit used for the aforesaid method. The present method adopts a combination strategy to combine Bevacizumab treatment with ultrasound exposure, which comprises at least the following steps: administering Bevacizumab to a subject; administering an ultrasound-response medium to said subject; and administering said subject with an ultrasound exposure. Specific effective amount of the Bevacizumab treatment and the ultrasound exposure were identified. The efficacy of conventional Bevacizumab treatment is significantly improved.

### CORRELATION APPLICATION

The application is based on a U.S. provisional application No. 62/253,805 filed on Nov. 11, 2015.

### BACKGROUND

### Description of Related Art

Glioblastoma multiforme (GBM) patients usually only have a 5-year survival rate under 5% and median overall survival of only 15 months after aggressive surgery and following adjuvant chemotherapy and radiation. Anti-angiogenic therapy is the focus of recent therapeutic development efforts because GBMs are highly vascularized tumors with irregular, extensive vascular proliferation, increased expression of angiogenic factors, and profoundly high levels of secreted vascular endothelial growth factor (VEGF). High GBM-associated VEGF expression results in growth and proliferation of endothelial cells, which correlates with tumor hypoxia and necrosis, and triggers tumor angiogenesis and progression.

Bevacizumab is a humanized monoclonal antibody specifically binds to the VEGF-A isoform and neutralizes endothelial cell proliferation. Bevacizumab reduces tumor neovascularization, improves blood vessel integrity, decreases tumor-associated edema, and improves clinical quality of life. Theoretically, consistent anti-angiogenesis drug administration should lead to vascular network destruction, impeding oxygen and nutrient transport, and ultimately causing tumor starvation. However, these anti-tumor effects are also impeded by “vascular normalization” mediated by the ability of anti-VEGF agents to effectively reduce vascular permeability and temporally reverse abnormal capillary leakage. The transient “vascular normalization” restores BBB integrity and restricts further bevacizumab penetration into brain parenchyma, thereby reducing angiogenic suppression of GBM cells and sustained tumor starvation to improve patient survival.

### SUMMARY

In light of the foregoing, one of the objectives of the present invention is to provide a method for treating brain cancer by using an anti-angiogenic therapy. Another objective of the present invention is to provide a kit for treating brain tumor. The method and the kit are able to provide better efficacy while using less dosage of anti-angiogenic medicine.

In order to achieve the aforesaid objectives, the present invention provides a method for reducing an effective amount of Bevacizumab required for treating brain tumor, comprising the following steps: administering Bevacizumab to a subject; wherein said Bevacizumab is of an amount of 0.011 to 11 mg/kg body weight; administering an ultrasound-response medium to said subject; and administering said subject with a ultrasound exposure of 0.1 to 2 MI (Mechanical index). Preferably, said ultrasound exposure is of 0.47 to 1.26 MI; more preferably, is of 0.55 to 0.84 MI.

Preferably, said Bevacizumab is of an amount of 0.11 to 11 mg/kg body weight. More preferably, said Bevacizumab is of an amount of 1.1 to 11 mg/kg body weight. Preferably, said administering for said Bevacizumab is via intravenous injection.

Preferably, said administering for said ultrasound-response medium is via intravenous injection. Preferably, said ultrasound-response medium is a plurality of particles. Preferably, said particles have a mean diameter of 0.1 to 10 μm. Preferably, said plurality of particles is of an amount of 1.9×10⁶ to 1.17×10⁸ particles/kg body weight for said administering. Preferably, said particles are microbubbles.

Preferably, said ultrasound exposure is administered on the central nervous system of said subject. Preferably, said brain tumor is Glioblastoma multiforme.

The present invention also provides a kit for treating brain tumor, comprising: a Bevacizumab formulation; an ultrasound-response medium; and a focus ultrasound system comprising an ultrasound transducer.

Preferably, said Bevacizumab formulation comprises 0.11 to 25 mg/ml of Bevacizumab and a carrier for injection; wherein said mg/ml is based on the total volume of said Bevacizumab formulation. Preferably, said carrier for injection is water, saline, polymer, emulsifier, surfactant or a combination thereof.

Preferably, said ultrasound-response medium comprises 1×10⁴ to 1×10¹² particle/ml of particles; wherein said particle/ml is based on the total volume of said ultrasound-response medium. Preferably, said particles have a mean diameter of 0.1 to 10 μm.

Preferably, said ultrasound-response medium is mixed with a carrier for injection. Preferably, said carrier for injection is water, saline, polymer, emulsifier, surfactant or a combination thereof.

Preferably, said brain tumor is Glioblastoma multiforme.

Preferably, said ultrasound system is a medical imaging based guidance ultrasound system. Preferably, said medical imaging comprises neuronavigation, ultrasonography, optical imaging, computed tomography, nuclear imaging (CT), or magnetic resonance imaging (MRI).

### BRIEF DESCRIPTION OF THE DRAWINGS

FIG. 1 shows the time course for experimental design in using ultrasound-induced BBB opening to enhance Bevacizumab delivery in glioma-bearing mice. Bevacizumab was given by IV at 50 mg/kg body weight weekly (days 1, 8, 15, 22, and 29 after 1st MRI screening), and tumor progression and survival were longitudinally followed by T2-MRI. Ultrasound exposure was conducted weekly (days 1, 8, 15, 22, and 29 after 1st MRI screening, before IV administration of Bevacizumab).

FIG. 2 illustrates the concepts and representative MRI observations of ultrasound induced blood-brain barrier (BBB) opening. (a) Conceptual diagram for experimental approach using ultrasound-induced blood-brain barrier opening to enhance Bevacizumab delivery for glioma treatment. (B to I) Representative CE-MRI analysis of ultrasound-induced BBB opening with intermediate (0.4 MPa, i.e. 0.63 MI) and aggressive (0.8 MPa, i.e. 2 MI) ultrasound exposure level. (b, c) Signal intensity level increase maps of contrast-enhanced T1-weighted images (in %); (d, e) area-under-the-curve (AUC) accumulation of R1 maps (in s⁻¹.min); (f, g) Ktrans maps (in min⁻¹); (h, i) Ve maps.

FIG. 3 shows the quantitation of delivered Bevacizumab penetration, correlation with MR imaging, and Western blotting. (a) Bevacizumab concentration with different ultrasound exposures (0, 0.4, and 0.8 MPa; i.e. 0, 0.63, and 2 MI respectively) level measured by HPLC; (b-e) Correlations of the Bevacizumab concentration with different indexes of contrast-enhanced MRI including SI increase in T1-WI (penal (b)), R1-AUC level change (penal (c)), Ktrans level change (penal (d)), and Ve level change (penal (e)). (f) Western blot showing enhanced Bevacizumab penetration into CNS after ultrasound-induced BBB opening. (g) Comparison of quantitative analysis of the Western blot intensity. The symbol “***” represents p<0.001. BEV=Bevacizumab.

FIG. 4 displays the fluorescent microscopic observations of ultrasound-assisted 70-kDa fluorescent-tagged dextrans delivery into CNS. (a, b) Left and right hemispheres of the controlled animal without ultrasound exposure and no fluorescent signal been detected due to the intact BBB. (c, d) animal treated with 0.3-MPa ultrasound exposure at the right hemisphere (fluorescent intensity of 42354±3937 a.u.). (e, f) animal treated with 0.4-MPa ultrasound exposure (i.e. 0.63 MI) at the right hemisphere (fluorescent intensity of 81329±7926 a.u.). (g, h) animal treated with 0.53 MPa ultrasound exposure (i.e. 0.84 MI) at the right hemisphere (fluorescent intensity of 85348±1050 a.u.). Bar=100 μm.

FIG. 5 displays the fluorescent microscopic observations of ultrasound-assisted 150-kDa fluorescent-tagged dextrans delivery into CNS. (a, b) Left and right hemispheres of the controlled animal without ultrasound exposure and no fluorescent signal been detected due to the intact BBB. (c, d) animal treated with 0.3 MPa/0.4-MHz ultrasound exposure at the right hemisphere (fluorescent intensity of 22716±3706 a.u.). (e, f) animal treated with 0.4-MPa ultrasound exposure (i.e. 0.63 MI) at the right hemisphere (fluorescent intensity of 29208±7518 a.u.). (g, h) animal treated with 0.53 MPa/0.4-MHz ultrasound exposure (i.e. 0.84 MI) at the right hemisphere (fluorescent intensity of 52643±11644 a.u.). Bar=100 μm.

FIG. 6 displays the fluorescent microscopic observations of ultrasound-assisted 250-kDa fluorescent-tagged dextrans delivery into CNS. (a, b) Left and right hemispheres of the controlled animal without ultrasound exposure and no fluorescent signal been detected due to the intact BBB. (c, d) animal treated with 0.3 MPa (i.e. 0.47 MI) ultrasound exposure at the right hemisphere (fluorescent intensity of 42012±5764 a.u.). (e, f) animal treated with 0.4 MPa ultrasound exposure (i.e. 0.63 MI) at the right hemisphere (fluorescent intensity of 60504.81±1956.97 a.u.). (g, h) animal treated with 0.53 MPa (i.e. 0.84 MI) ultrasound exposure at the right hemisphere (fluorescent intensity of 71672±7770 a.u.). Bar=100 μm.

FIG. 7 shows the comparison of the fluorescence intensity change under various triggered pressure and molecular sizes enhanced by ultrasound. Control animal without receiving ultrasound exposure do not allow dextrans penetration due to the blockage of BBB. Higher fluorescence intensity can be detected when higher acoustic pressure was applied or smaller size of fluorescent-tagged dextrans been administered.

FIG. 8 indicates the correlations of the fluorescence intensity with the concentrations of three fluorescent-tagged dextrans. (a-c) Various size of fluorescent-tagged dextrans all show high correlation between the fluorescence intensity to the concentration (r2=0.9517, 0.8447, and 0.9679 for 70-kDa (penal (a)), 150-kDa (penal (b)), and 250 k-Da (penal (c)), respectively).

FIG. 9 displays the representative T2-weighted MRI to follow tumor progression. (a-d) T2-weighted MRI was employed to monitor brain tumor progression by weekly from days 7 to 35 in each experimental group. Ultrasound-induced BBB opening alone (penal (b)) presented similar tumor progression to the untreated control (penal (a)), whereas bevacizumab administration alone provided improved tumor progression suppressed (penal (c)). Among groups, combined ultrasound-induced BBB opening with bevacizumab administration provided the most significant tumor-suppressing effect (penal (d)).

FIG. 10 shows the tumor progression and animal survival analysis. (a, b) Tumor progression from days 7 to 35 in each experimental groups. Penal (a) shows tumor volume (in mm3) and penal (b) shows tumor weekly progression rate (in %). (c) Kaplan-Meier plot to demonstrate animal survival among each experimental groups. BEV=Bevacizumab.

FIG. 11 shows the comparison of HE-stains and CD-31-stained immunochemistry (IHC) fluorescent microscopy. (a-c) HE-stain and CD-31 IHC microscopy show brain tumor treated with bevacizumab administration alone. (d-f) HE-stain and CD-31 IHC microscopy show brain tumor treated with combined ultrasound-induced BBB opening with bevacizumab administration. Bar=500 (g) Quantitative comparison of the vessel density measured within the tumor regions. (h, i) Zoomed images show CD-31 labeled vascularature obtained from (C) and (F), respectively. Bar=100 μm.

FIG. 12 shows the represented decay-corrected PET/CT images of BBB-opening mice at 15 min post-injection of ⁶⁸Ga-bevacizumab. a. Experimental group (n=3), mice were treated with ultrasound and injected with 68Ga-bevacizumab. Circles indicate the ultrasound targeting sites. Control group (n=3) with the same dose injection of ⁶⁸Ga-bevacizumab, but without the ultrasound treatment. b. PET quantification of BBB opening effect, and bio-distribution profile of major organs (Heart, Liver, kidney, Lung, Muscle) among both groups.

FIG. 13 shows the quantitation of delivered Bevacizumab penetration. “Blank” represents the control group (untreated group). “50 mg”, “30 mg” and “10 mg” represent the groups treated with 50 mg/kg body weight, 30 mg/kg body weight and 10 mg/kg body weight of bevacizumab respectively without ultrasound exposure. “Ultrasound+50 mg”, “ultrasound+30 mg” and “ultrasound+10 mg” represent the groups treated with 50 mg/kg body weight, 30 mg/kg body weight and 10 mg/kg body weight of bevacizumab respectively with ultrasound exposure.

FIG. 14 shows the progress of tumor by volume. “Control” represents the control group (untreated group). “50 mg” represents the group treated with 50 mg/kg body weight of bevacizumab without ultrasound exposure. “ultrasound+30 mg” and “ultrasound+10 mg” respectively represent the groups treated with 30 mg/kg body weight or 10 mg/kg body weight of bevacizumab with ultrasound exposure.

FIG. 15 demonstrates the survival probability of mice model experiment. “Control” represents the control group (untreated group). “50 mg” represents the group treated with 50 mg/kg body weight of bevacizumab without ultrasound exposure. “ultrasound+30 mg” and “ultrasound+10 mg” respectively represent the groups treated with 30 mg/kg body weight or 10 mg/kg body weight of bevacizumab with ultrasound exposure.

### DETAILED DESCRIPTION

Low-pressure burst-mode transcranial ultrasound exposure can locally, temporally and reversibly open the BBB. Transcranial ultrasound exposure is capable of noninvasive delivery of focal energy to deep-seated brain locations. BBB disruption can increase the local concentration of therapeutic agents in the brain without damaging normal tissue.

The term “effective amount” used herein is referred to an amount of reagent, drug or medicine that is sufficient to obtain the desired effect for the subject in need. The description of “treating brain tumor” or alike used herein is referred to but is not limited to reduce and/or prevent from the progress of the tumor, to reduce the size of the tumor, to reduce and/or prevent from the effect of the tumor on the normal physiology of a subject, or a combination thereof.

The term “ultrasound-response medium” used herewith is referred to as a medium which is able to provide cavitation in response to the acoustic power of ultrasound. In a preferable embodiment, said cavitation is able to open the BBB and more preferably to increase the permeability across the BBB. Without being bound by theory, said ultrasound-response medium is used synergically with said ultrasound exposure to open or disrupt the BBB and thereby increasing the permeability of medicine.

One aspect of the present invention is to provide a method for enhancing the efficacy of Bevacizumab in treating brain tumor. By using the present method, the effective amount of Bevacizumab required for treating brain tumor can be reduced. In an alternative embodiment, said brain tumor could be meningiomas or astrocytomas. In a specific embodiment, said astrocytomas is Glioblastoma multiforme.

The method of the present invention comprises administering Bevacizumab to a subject; administering an ultrasound-response medium to said subject; and administering said subject with an ultrasound exposure. In an alternative embodiment, there is no particular order for conducting the three steps above.

In a preferable embodiment, said amount of Bevacizumab for administering is for human being. In an alternative embodiment, said amount of Bevacizumab for administering is calculated from the data obtained from a mouse model experiment. For instance, the calculation is based on the formula taught in Reagan-Shaw et al., FASEB J. 2008 March; 22(3):659-61. In an embodiment, said Bevacizumab is of an amount of 0.011 to 11 mg/kg body weight; preferably, is of 0.11 to 11 mg/kg body weight; more preferably, is 1.1 to 11 mg/kg body weight.

In an alternative embodiment, said ultrasound-response medium is a plurality of particles. In a preferable embodiment, an amount of 1.9×10⁶ to 1.17×10⁸ particles/kg body weight of said particles are administered. In an alternative embodiment, said particles have a mean diameter of 0.1 to 10 μm.

Preferably, said particles are microbubbles. In an alternative embodiment, said microbubble has a core-shell structure; wherein said shell is made of biocompatible materials (including but not limited to albumin, lipid, or polymer alike) and the core is a biocompatible gaseous medium. In an alternative embodiment, said microbubble comprises albumin-coated microbubble, lipid-shelled microbubble, gas-filled microbubble, or a combination thereof. In another alternative embodiment, said microbubble may be commercially available product, such as products of SonoVue®, Definity®, Optison®, Imagent®, Levovist®, or Lumason®.

In an alternative embodiment, said ultrasound exposure is of 0.1 to 2 MI (Mechanical index; defined as P/Ad, where P is peak negative pressure (in MPa) and f is frequency (in MHz)); preferably is of 0.47 to 1.26 MI; more preferably is of 0.55 to 0.84 MI. Preferably, said ultrasound exposure is conducted on the central nervous system of the subject; more preferably, on the brain of the subject.

Another aspect of the present invention is a kit for treating brain tumor. In an alternative embodiment, said kit is used for practicing the method of the present invention. In an alternative embodiment, said brain tumor could be meningiomas or astrocytomas. In a specific embodiment, said astrocytomas is Glioblastoma multiforme. The kit of the present invention comprises a Bevacizumab formulation; an ultrasound-response medium; and an ultrasound system. In an alternative embodiment, said Bevacizumab formulation comprises 0.11 to 25 mg/ml of Bevacizumab and a carrier for injection. Said carrier for injection could be but not limited to water, saline, polymer, emulsifier, surfactant or a combination thereof.

Said ultrasound-response medium of the kit of the present invention is the same as set forth in the preceding paragraphs regarding the method of the present invention. In a preferable embodiment, said ultrasound-response medium is mixed with a carrier for injection as a formulation and said formulation comprises 1×10⁴ to 1×10¹² particle/ml of particles; wherein said particle/ml is based on the total volume of said formulation. Alternatively, said carrier for injection is water, saline, polymers, emulsifier, surfactant or a combination thereof. In a preferably embodiment, said particles are formulated as a preparation comprising said particles, saline, and heparin.

A typical ultrasound system usually includes an ultrasound transducer and a water tank. In a specific embodiment, said ultrasound system is a medical imaging based guidance ultrasound system. In a preferable embodiment, said ultrasound system is a neuronavigation-guided ultrasound system, which further includes a neuronavigation system (Tsai et al, Ultrasonics Symposium (IUS), 2015 IEEE International). In an alternative embodiment, said ultrasound system could be an ultrasonography-guided ultrasound system, an optical imaging-guided ultrasound system, a computed tomography-guided ultrasound system, a nuclear imaging (CT)-guided ultrasound system, or a MRI-guided ultrasound system.

The following experiments and details are provided for further explanation to the spirit and conception of the present invention. It shall be noted that the following information does not intend to limit the claim scope of the present invention. Those having ordinary skill in art would be able to make modification to the following detailed description of the present invention without apart from the spirit of the present invention.

### Experiments 1 to 4

### Materials and Methods of the Following Experiments 1 to 4

### 1. U87 Glioma Animal Model.

U87 mouse glioma cells were cultured at 37° C. with 5% CO₂ in MEM with 10% fetal bovine serum and 1% penicillin/streptomycin (Invitrogen). Pathogen-free male NU/NU mice (5-7 weeks old, 20-25 gram) from BioLASCO (Taiwan) were housed in a controlled environment and all experiments were approved by our Institutional Animal Care and Use Committee. To implant U87 cells, animals were anesthetized with 2% isoflurane gas and immobilized on a stereotactic frame. A sagittal incision was made through the skin overlying the calvarium, and a 27G needle was used to create a hole in the exposed cranium 1.5 mm anterior and 2 mm lateral to the bregma. Five microliters of U87 cell suspension (1×10⁵ cell/μl) was injected at a depth of 2 mm from the brain surface over a 5-minute period, and the needle was withdrawn over 2 minutes. Xenograft growth was monitored by MRI for 10 days post implantation.

### 2. Experimental Design.

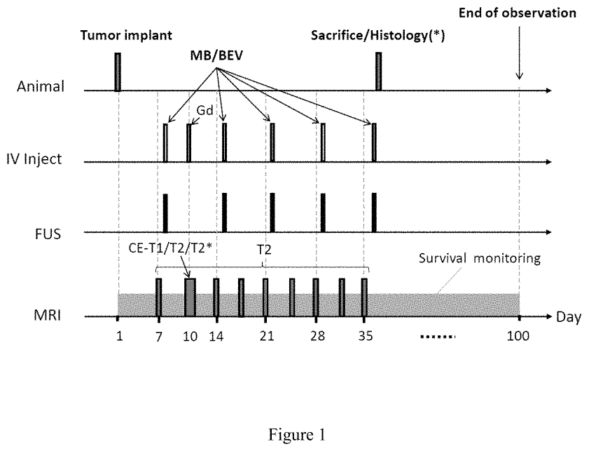

Twelve normal and 42 tumor-bearing mice were used. In group 1, the aim was to assess if ultrasound-induced BBB opening enhanced bevacizumab penetration in normal mice (n=4), and to determine the appropriate ultrasound power range (n=4) and for Western blot analysis (n=8; 4 normal and 4 tumor-bearing). In group 2, the aim was to evaluate the therapeutic efficacy of bevacizumab combined with ultrasound exposure in tumor-bearing mice. There were 4 sub-groups: (1) sham (n=7); (2) ultrasound alone (n=9); (3) bevacizumab alone (n=6), bevacizumab was given by IV at 50 mg/kg body weight weekly (days 1, 8, 15, 22, and 29 after 1st MRI screening); and (4) bevacizumab+ultrasound-BBB opening (n=12), bevacizumab was given by IV at 50 mg/kg body weight weekly (days 1, 8, 15, 22, and 29 after 1st MRI screening). In group 2, the endpoint was MRI-measured tumor volume>200 mm³ or higher than 20% body weight drop during one week. The time course for the experimental design is shown in FIG. 1.

### 3. Ultrasound Exposure.

The ultrasound is a neuronavigation-guided ultrasound according to Tsai et al, Ultrasonics Symposium (IUS), 2015 IEEE International. An ultrasound transducer (Sonic Concepts Inc., Washington, USA; diameter=60 mm, radius of curvature=52 mm, frequency=400 kHz) was applied to generate concentrated ultrasound energy. An arbitrary function generator (33220A, Agilent, Palo Alto, Calif.) was used to produce the driving signal, which was fed to a radio frequency power amplifier (No. 500-009, Advanced Surgical Systems, Tucson, Ariz.) operating in burst mode. Anesthetized animals were immobilized on a stereotactic frame and a PE-10 catheter was inserted into tail veins. The animal was placed directly under an acrylic water tank with the head firmly attached to a 4×4 cm′ thin-film window at the bottom to allow entry of ultrasound energy. SonoVue® SF₆-filled ultrasound particles (2-5 μm, 10 μl/mouse, 2×10⁸ microbubble/ml; Bracco, Milan, Italy) were administered intravenously before ultrasound. The tumor-implant hemisphere brain site was then exposed to burst-tone mode ultrasound (electrical power=4-18 W; peak negative pressure=0.1 to 2 MI; burst length=10 ms; pulse repetition frequency=1 Hz; exposure time=60 s).

### 4. Magnetic Resonance Imaging (MRI).

Contrast-enhanced MRI was used to assess increased BBB permeability via Gd-DTPA administration (Magnevist, Wayne, N.J., USA), and obtain the following imaging indexes: (1) signal intensity increase in T1-weighted images after Gd-DTPA injection 10; (2) Area-under-the-curve of the R1 relaxivity over a period (denoted as R1-AUC); (3) the kinetic parameter “Ktrans”, transfer rate constant from the intravascular system to the extracellular extravascular space, characterized from Kety's compartment model; (4) the kinetic parameter “Ve”, volume fraction of the contrast agent in the EES, characterized from Kety's compartment model.
Susceptibility-weighted imaging sequences were acquired to identify large-scale erythrocyte extravasations (parameters: TR/TE=30 ms/18 ms; flip angle=40°; slice thickness=0.6 mm; matrix size=256×384; and FOV=80×130 mm²). MRI images were acquired on a 7-Tesla magnetic resonance scanner (Bruker ClinScan, Germany) and a 4-channel surface coil was used on the top of the mouse brain. Anesthetized animals were placed in an acrylic holder and positioned in the magnet center. Tumor size was quantified using turbo-spin-echo based T2-weighted images with the following parameters: pulse repetition time (TR)/echo time (TE)=2540/41 ms; FOV=19×30 mm² (156×320 pixels); slice thickness=0.5 mm; flip angle=180; total acquisition time=155 seconds. Relative tumor size was estimated by measuring the single image slide containing the maximum tumor area, and animals were longitudinally imaged every 7 days for up to 35 days after the first MRI screen.

### 5. Histological Examination.

Evans Blue dye was injected intravenously and animals were sacrificed after two hours. Histopathology was performed on 10-μm sections from paraformaldehyde-fixed, paraffin-embedded brains. Slides were placed in a staining jar containing a hydrochloric acid-potassium ferrocyanide solution for 30 minutes at room temperature. The slides were counterstained by nuclear fast red for 5 min. Hematoxylin and eosin (H&E) staining was used to evaluate brain tissue damage and tumor progression.
To assess vascularity, slides were subjected to immunofluorescence with anti-mouse-CD31 antibody (1:500, 550274, BD Pharmingen, NJ, USA) and goat-anti-rat Dylight 488 antibody (1:200, 405409, Biolegend, CA, USA). Tumor blood vessels were quantified as total vessel area relative to tumor cross-sectional area.

### 6. Western Blot.

50 μg protein (goat-anti-Human IgG, AP112P, Millipore, USA) from homogenized brain tissues was used for Western blot analysis. Band optic density on film was analyzed using the BioSpectrum Imaging System (UVP LLC, Upland, Calif.).

### 7. Quantitative Bevacizumab Analysis.

Animals were sacrificed 2 hours after ultrasound and brains were divided into left and right hemispheres and homogenized with 10 μl/mg mL methanol. Extracted Bevacizumab was analyzed by HPLC with a Model L-2400 UV detector and Model L-2130 pump (Hitachi), and a SUPELCOSIL™ LC-18 column (4.6□ 250 mm).

### 8. Statistical Analysis.

Statistical significance was calculated using either two-tailed unpaired t test or Mantel-Cox test. The Kaplan-Meier method was used for survival analysis. Statistical significance was assumed at p<0.05.

### Experiment 1: Ultrasound Effectively Opens BBB and Increases the Concentration of Bevacizumab in the Brain

In this experiment, transcranial ultrasound exposure of 0.63 MI or 2 MI (i.e. with a negative peak pressure of 0.4 MPa or 0.8 MPa) was performed in accordance with the method set forth above to evaluate its effect on opening the BBB. Also, bevacizumab was administered before the ultrasound exposure was performed to see if the ultrasound exposure contributed any benefit to the penetration of Bevaczumab through the BBB.

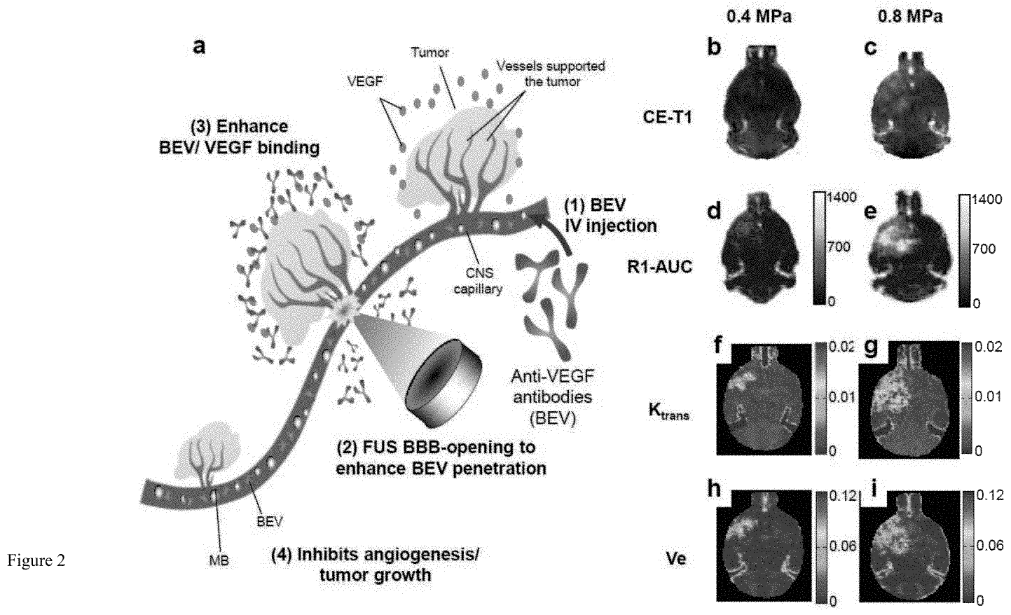

The opening of BBB was confirmed by contrast-enhanced MRI. Four types of CE-MRI indexes were used for this confirmation. The result shown in FIG. 2 indicated that both of 0.63 MI and 2 MI ultrasound exposure effectively opened the BBB at target brain regions. Moreover, higher pressure contributed to greater BBB-opening effect.

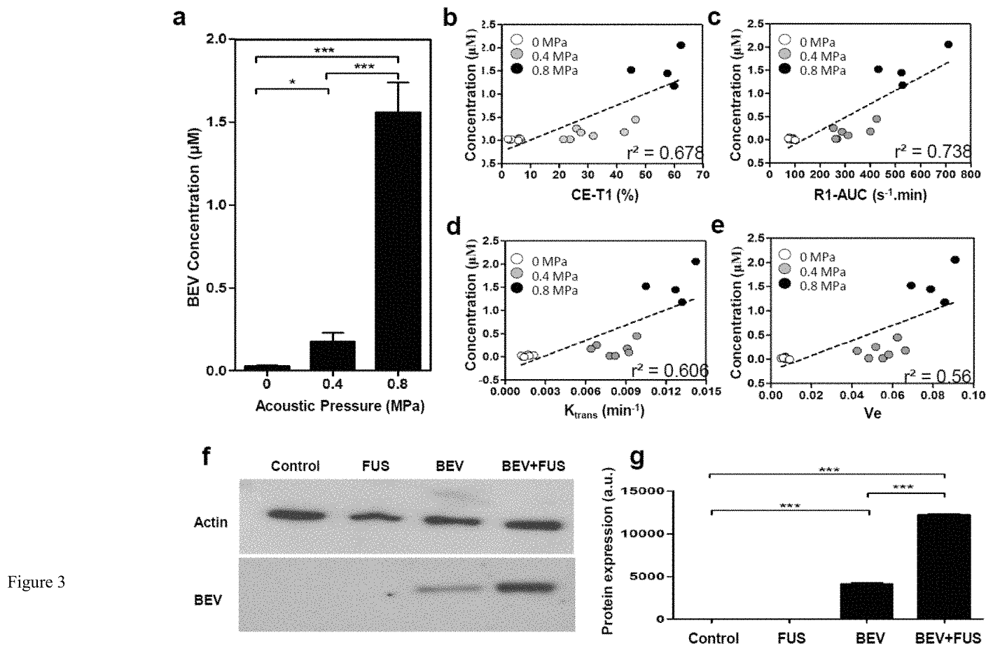

HPLC was used to quantitate bevacizumab concentration in the brain. The result showed that ultrasound increased bevacizumab concentration in CNS parenchyma. The intermediate 0.63 MI ultrasound exposure resulted in 0.175±0.15 μM of bevacizumab penetration into CNS (i.e., 5.73-fold increase), whereas aggressive 2 MI ultrasound exposure significantly increased bevacizumab to 1.554±0.37 μM (i.e., 58.77-fold increase) (FIG. 3a). In controls, bevacizumab is normally limited to the circulation, and penetrated the CNS in very limited amounts (0.026±0.02 μM). The four CE-MRI indices with correlations to bevacizumab concentrations were also analyzed (FIGS. 3b-e). R1-AUC was observed to have the highest linear correlation with the measured bevacizumab level (FIG. 3c; r²=0.738). Either increased signal intensity in T1-weighted images or Ktrans provided a moderate correlation to bevacizumab level with a correlation coefficient higher than 0.6 (r²=0.678 and 0.606, respectively; FIGS. 3b and 3d), whereas Ve was least correlated with the bevacizumab level (r²=0.56; FIG. 3e). Overall, the four indices provided sufficient correlation with the in vivo ultrasound-induced BBB opening enhancement of CNS bevacizumab delivery.

### Experiment 2: The Effect of Ultrasound-Induced BBB Opening on Molecular Penetration of Different Molecular Size

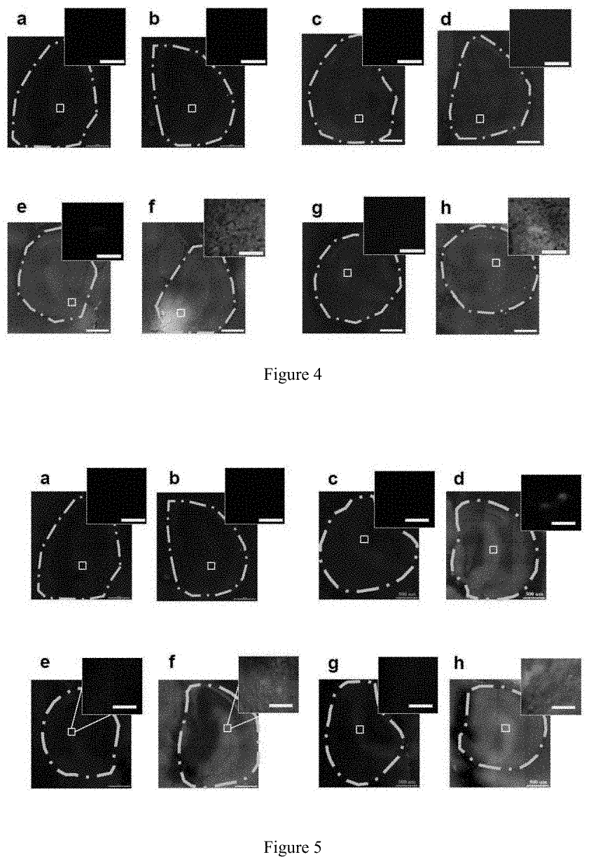

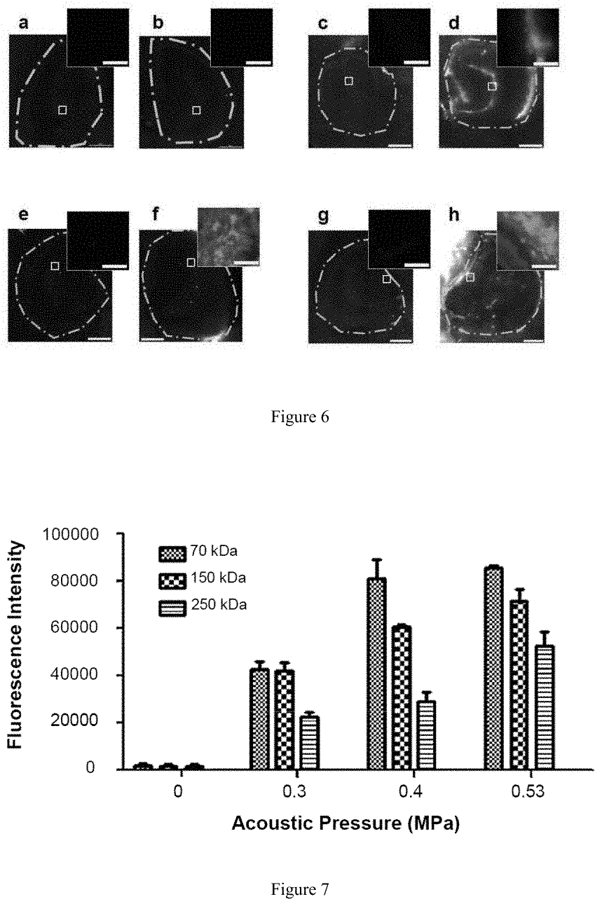

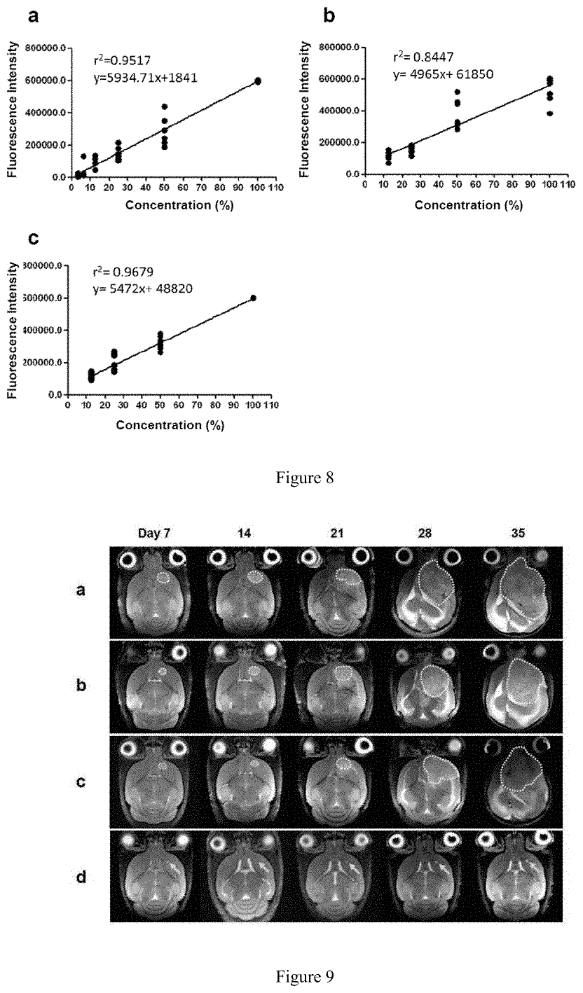

To understand the influence of molecular penetration with the molecular size and ultrasound exposure level, fluorescent-tagged dextrans (70 to 250 kDa) with similar molecular weight to bevacizumab (150 kDa) were served as surrogates to fine-tune the exposure level (0.55 to 0.84 MI) around the intermediate exposure level (i.e., 0.4 MPa/0.63 MI). Pressure level of above 0.55 MI were found to able to induce BBB opening and allow penetration of all sizes of molecule, including the 70-kDa (FIG. 4), 150-kDa (FIG. 5), and 250-kDa (FIG. 6) fluorescent-tagged dextrans, suggesting 0.63 MI exposure level should provide stable and consistent molecular penetration with the molecule at least up to 250 kDa. Yet, the fluorescent intensity increased in the brain either increasing the ultrasound exposure level or decreasing the molecular weight, suggesting smaller molecular or higher exposure level contributes to higher molecular penetration (FIG. 7). Ultrasound exposure increased to 0.84 MI began to induce noticeable but minimal extravasations of erythrocytes (FIG. 8c), but not observed at lower exposure level (FIGS. 8a-b). These extravasations caused by 0.84 MI exposure was unable to be detected in T2*MRI; in contrast, 2 MI exposure induced apparent drop in signal intensity in T2*MRI, suggesting a relatively larger scale of extravasations of erythrocytes been induced than 0.84 MI exposure. Considering together, the 0.63 MI ultrasound exposure level was selected for the following glioma-bearing tumor sonication.

Western blots were performed to measure bevacizumab protein levels in ultrasound-exposed brains (FIG. 3f). No bevacizumab was detected in either control or ultrasound-alone animals. With bevacizumab administration alone, only a 150 kDa band was detected, indicating the presence of bevacizumab in the CNS. In contrast, the combination of ultrasound exposure (0.63 MI) with bevacizumab administration markedly increased the levels of detectable bevacizumab protein. Ultrasound with bevacizumab administration produced a nearly 3-fold increase in hybridization intensity (FIG. 3g), which was similar to the improved penetration determined by HPLC analysis (12,238 versus 4,138 in detected counts).

### Experiment 3: Ultrasound and Bevacizumab Combination Strategy for Glioma Therapy

We aimed to evaluate whether the enhanced bevacizumab delivery with ultrasound-induced BBB opening improved glioma therapy. The ultrasound exposure level of 0.63 MI was selected to avoid accompanying erythrocyte extravasation for the planned five-week repetitive treatment regimen. Please see the “Experiment design” section and FIG. 1.

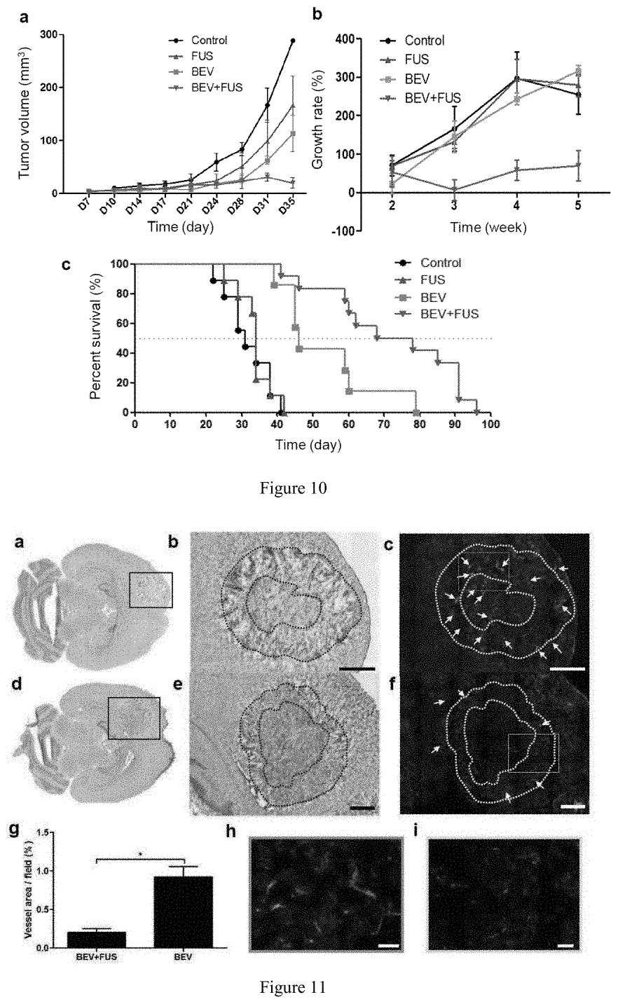

Typical tumor follow-up images are shown in FIG. 9 (days 7 to 35), with the tumor volume progression measured by T2-MRI (FIGS. 10a-b). Overall, the untreated controls (FIG. 9a) and ultrasound-exposure alone (FIG. 9b) showed progressive tumor growth (tumor progression ratios with follow-up interval of 10-35 days were 172±33.2% and 185±22.6%, respectively). Treatment with bevacizumab alone (FIG. 9b) showed a tumor suppressive effect in the first four treatment weeks, but tumor progression surged at week 5. The overall tumor progression (152±27.7%) was not significantly different from the control (p=0.6477) or the ultrasound-alone treatment (p=0.3735) groups. Bevacizumab administration combined with ultrasound-BBB opening (FIG. 9d) consistently reduced tumor progression throughout the five-week treatment regimen, and showed the most profound control of tumor progression when evaluated via T2-MRI (45±16.11%, p=0.0016 vs. control group; FIGS. 10a-b). This result suggests that enhanced bevacizumab penetration facilitated by ultrasound exposure contributes to a stable and sustained anti-angiogenic effect to control tumor progression.

The combined ultrasound and bevacizumab treatment was conducted for 5 weeks (days 7-35), then animal survival at 100 days evaluated (FIG. 10c and Table 1). ultrasound-only animals did not show effective extension of survival, the same as observed in MRI-monitored tumor progression (median survival=34 days when compared to 31 days in control; ISTmedian=9.68%; p=0.5407). Five-week bevacizumab administration contributed to significantly prolonging survival to 46 days compared to controls (ISTmedian=48.39%; p=0.0369). The combined ultrasound-induced BBB opening and bevacizumab administration strategy resulted in significantly improved median survival of 73 days, which is 2.35-fold higher than the median survival of control and 1.58-fold higher than bevacizumab alone. The equivalent observation also holds when mean survival and ISTmean were calculated.

TABLE 1
Summary of animal survival analysis. The increase in median survival time (ISTmedian;
in %) was found to be 135% in combined ultrasound/bevacizumab group and outperformed
to 48% in bevacizumab-alone group. Similarly, the increase in mean survival time (ISTmean;
in %) was found to be 143% in combined ultrasound/bevacizumab group and outperformed
to 76% in bevacizumab-alone group; p-values are all relative to the control group.

| | Median survival | IST_{median} | Mean survival | ISTₘₑₐₙ | |
|---|---|---|---|---|---|
| Group (n) | (days) | (%) | (days) | (%) | p-value |
| Control (7) | 31 | — | 29.71 ± 5.35 | — | — |
| Ultrasound alone (9) | 34 | 9.68 | 33.67 ± 4.82 | 13.33 | 0.5407 |
| Bevacizumab alone (6) | 46 | 48.39 | 52.33 ± 14.8 | 76.14 | 0.0369 |
| Ultrasound + Bevacizumab (12) | 73 | 135.48 | 72.33 ± 18.9 | 143.45 | 0.0001 |

H&E staining and CD-31 immunohisochemistry (IHC) were used to assess morphological changes and vascular distributions of week-4 tumor xenografts after either administration of bevacizumab alone or combined with ultrasound-induced BBB opening (FIG. 11). Similar tumor morphology was detected in bevacizumab-alone or bevacizumab+ultrasound-BBB opening: a necrotic tumor core accompanied by highly proliferative tumor rims. CD-31 IHC visualized an enriched vascular structure within the tumor rims, but not the necrotic core, of tumors treated with bevacizumab-alone (marked by white arrows). In contrast, a significant reduction in the CD-31 marked vascular distribution was observed in bevacizumab+ultrasound treated xenografts, and tumor rims were significantly smaller in dimension than tumors treated with bevacizumab alone (contoured by dashed line; FIG. 11c and FIG. 11f). Quantitative analysis of tumor blood vessels demonstrated that the mean (±SD) percent vessel area in tumor xenografts of animals treated with bevacizumab alone was 0.93±0.19%. The vessel area was markedly decreased in xenografts treated with combined ultrasound and bevacizumab, with a vessel area of 0.2±0.07% (FIG. 11c, FIG. 11f, FIG. 11g). Tissue necrosis was also observed by H&E staining in the area surrounding the tumor (FIG. 11b, FIG. 11e). These data suggest that addition of ultrasound-BBB opening enhanced bevacizumab penetration and effectively suppressed angiogenesis, particularly in the tumor periphery, resulting in improved tumor control.

### Experiment 4

### Experiment 4: Ultrasound-Induced BBB Opening Increases the Penetration of Bevacizumab

We had shown that the ultrasound increased the molecular penetration of different molecular size in previous experiment. It was further proposed that the ultrasound exposure can decrease the required dosage of bevacizumab in treating giloblastoma multiforme. In order to verify our hypothesis, normal mice and bearing mice were treated with bevacizumab of various dosages with or without ultrasound. The bevacizumab concentration in CNS parenchyma was determined by HPLC. Penetration curve (given dosage vs. bevacizumab concentration in CNS parenchyma) was made accordingly. The operations of ultrasound and HPLC were the same as set forth in the previous experiments 1-4.

Specifically in the experiments, 42 normal mice and 42 tumor-bearing mice were used. In group 1, mice were given bevacizumab of 50 mg/kg body weight alone (n=3) or with ultrasound (n=3); in group 2, mice were given bevacizumab of 25 mg/kg body weight alone (n=3) or with ultrasound (n=3); in group 3, mice were given bevacizumab of 10 mg/kg body weight alone (n=3) or with ultrasound (n=3); in group 4, mice were untreated (n=3) as negative controls. The bevacizumab concentration can also be determined via microPET/micro-CT. The following is the primary result of evaluating the dynamic change of bevacizumab distribution via microPET/micro-CT.

Dynamic change of bevacizumab distribution was evaluated via microPET/micro-CT fused imaging with bevacizumab been radiolabeled with radioisotope ⁶⁸Ga³⁺ (half-life: 68 min; procedures see the supplementary methods). The PET images were acquired 15 minutes after intravenous injection and the representative coronal PET images of both groups, with this time point the contrast of targeting region reached maximal as the nonspecific binding.

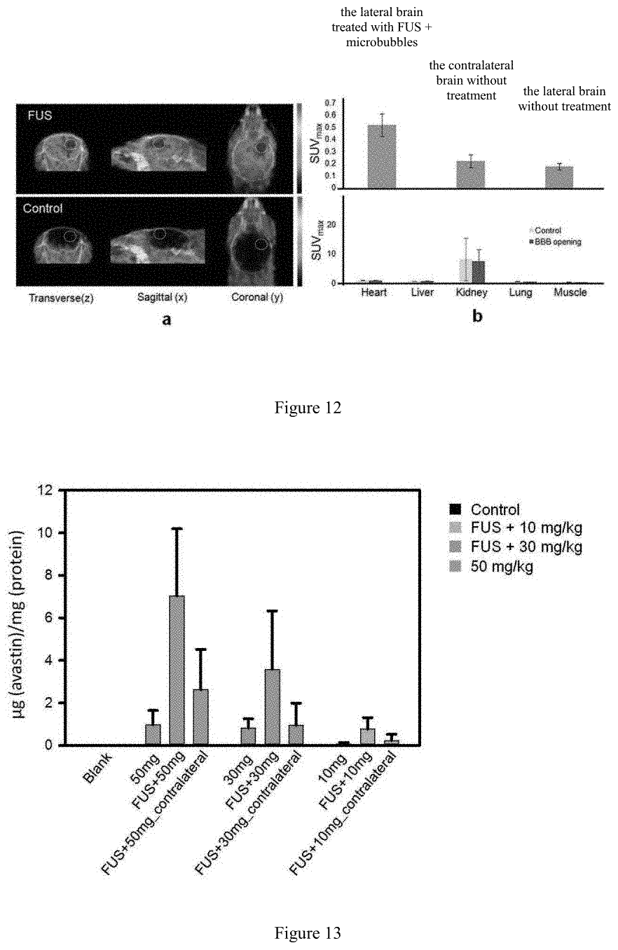

The ⁶⁸Ga-bevacizumab penetration in BBB opening mice has shown significant enhancement in the insonified region by using ultrasound treatment combined particles administration. The penetration of 68Ga-bevacizumab increased from 0.16±0.026 SUVmax to 0.64±0.09 SUVmax, (FIG. 12a) corresponding to a 4-fold enhancement, which was comparable to the HPLC quantitated results (5.73-fold). Systemic uptake shows that bevacizumab majorly absorbed and metabolized from kidney. Animals underwent ultrasound-BBB opening do not change the metabolized route of bevacizumab, although there was a slightly SUVmax level decrease when compared to the control groups (7.53±4.05 SUVmax v.s. 8.19±7.29 SUVmax; p>0.05; see FIG. 12b).

### Experiment 5

### Materials and Methods of the Following Experiment 5:

### 1. Experimental Design

A total of 45 tumor-bearing mice were used, including normal (n=19) and tumor-bearing mice (n=26). Experiments were divided into two groups. In experimental group 1, the primary aim was to assess if ultrasound-induced BBB opening promoted Bevacizumab penetration and deposition in brain tissue (n=19). The majority of normal animals were divided into two groups, without ultrasound-induced BBB opening (n=10) and with ultrasound-induced BBB opening (n=9), and the brain samples with the Bevacizumab concentration were quantified using enzyme-linked immunosorbent assay (ELISA) kit. All animals were sacrificed and the brain samples were preserved 2 hours after Bevacizumab administration.
In experimental group 2, the aim was to evaluate the therapeutic efficacy of Bevacizumab combined with ultrasound in tumor-bearing mice. Bevacizumab uptake was performed 10 days after U87 glioma cell implantation. Bevacizumab was IV administered five times (days 10, 17, 24, 31 and 38 after U87 glioma cell implantation), and the tumor progression and survival were both longitudinally followed. Animals were divided into 4 sub-groups: (1) sham (without Bevacizumab administration) (n=10); (2) Bevacizumab 10 mg/kg per week for 5 weeks plus ultrasound-induced BBB opening (n=8); (3) Bevacizumab 30 mg/kg per week for 5 weeks plus ultrasound-BBB opening (n=12) and (4) Bevacizumab 50 mg/kg per week for 5 weeks (n=6).

### 2. Ultrasound Parameter

The ultrasound is a neuronavigation-guided ultrasound according to Tsai et al, Ultrasonics Symposium (IUS), 2015 IEEE International. An ultrasound transducer (Imasonics, Besancon, France; diameter=60 mm, radius of curvature=80 mm, frequency=400 kHz) was applied to generate concentrated ultrasound energy. An arbitrary function generator (33120A, Agilent, Palo Alto, Calif.) was used to produce the driving signal, which was fed to a radio frequency power amplifier (No. 500-009, Advanced Surgical Systems, Tucson, Ariz.) operating in burst mode. Animals were anesthetized with 2% isoflurane and immobilized on a stereotactic frame. The top of the cranium was shaved with clippers, and a PE-10 catheter was inserted into the tail vein. The animal was placed directly under an acrylic water tank (with a window of 4×4 cm² at its bottom sealed with a thin film to allow entry of the ultrasound energy) with its head attached tightly to the thin-film window. SonoVue SF₆-filled ultrasound particles (2-5 μm, 10 μl/mouse, 2×10′ microbubble/ml; Bracco, Milan, Italy) were administered intravenously before treatment. The tumor implanted brain site was then exposed to burst-tone mode ultrasound to locally open the BBB (0.63 MI; burst length=10 ms; pulse repetition frequency=1 Hz; exposure time=60 s).

### 3. Mouse Glioma Model

U87 mice glioma cells were cultured at 37° C. in a humidified 5% CO₂ atmosphere in minimum essential median (MEM) supplemented with 10% fetal bovine serum and 1% penicillin/streptomycin (Invitrogen). Cells were harvested by trypsinization, washed once with phosphate-buffered saline (PBS), and resuspended (1.6×10⁵ cell/μl) in MEM for implantation into the striatum of mouse brains. Pathogen-free male NU/NU mice (5 to 7 weeks old) were purchased from BioLASCO (Taiwan). Mice were housed and maintained in a controlled environment and all procedures were performed in accordance with the experimental animal care guidelines of the Animal Committee of Chang Gung University. To implant U87 tumor cells, we anesthetized animals with 2% isoflurane gas and immobilized them on a stereotactic frame. A sagittal incision was made through the skin overlying the calvarium, and a 23G needle was used to create a hole in the exposed cranium 2 mm anterior and 2 mm lateral to the bregma. Three microliters of U87 glioma cell suspension were injected at a depth of 2 mm from the brain surface. The injection was performed over a 3-minute period, and the needle was withdrawn over another 2 minutes. The growth of the mouse brains was monitored by MRI for 10 days post tumor cell implantation.

### 4. Quantitative Bevacizumab Analysis

Brain tissues were collected after 2 hours after the administration of Bevacizumab. The brain tissues were homogenized and centrifuged at 5000 rpm for 10 minutes until a clear separation between supernatant fluid and the tissue components was seen. The concentration of Bevacizumab was measured with an enzyme-linked immunosorbent assay (ELISA) kit (My Biosource, Inc. San Diego, Calif., USA), The optical density was determined at 450 nm with the absorption spectrophotometer with the background subtraction at 620 nm.

### Experiment 5: Ultrasound Increases the Penetration of Bevacizumab and Promote the Efficacy of Bevacizumab in Treating Glioblastoma Multiforme

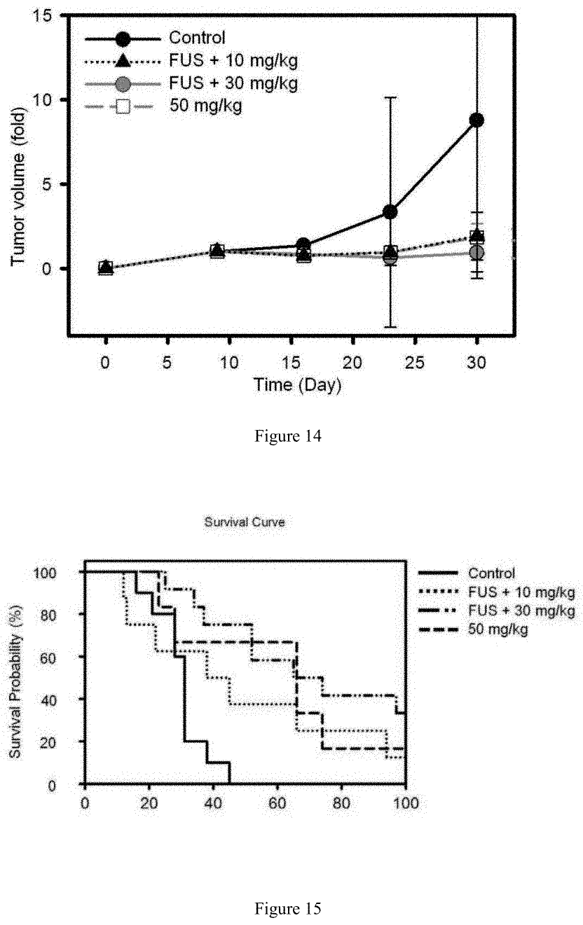

According to the result shown in FIG. 13, it was noted that, basically, higher dosage of Bevacizumab administered resulted in higher penetration of Bevacizumab in the brain. Furthermore, the treatment of 10 mg/kg body weight of Bevacizumab with ultrasound exhibited comparable concentration of Bevacizumab in the brain sample with the treatment of 50 mg/kg body weight of Bevacizumab without ultrasound exposure. The treatment of 30 mg/kg body weight of Bevacizumab with ultrasound showed much more Bevacizumab penetration than the treatment of 50 mg Bevacizumab without ultrasound exposure. The result demonstrated that, with the assistance of ultrasound, less amount of Bevacizumab was required to achieve comparable concentration in the brain. Moreover, the tumor progression was moderated in the groups with treatment in comparison with the control group. In addition, the groups with ultrasound exposure exhibited results though less amount of Bevacizumab was administered (FIG. 14).

Further in the survival test (FIG. 15), among others, the treatment of 30 mg/kg body weight of Bevacizumab with ultrasound contributed the best survival probability. The survival probability of the group treated with 10 mg/kg body weight of Bevacizumab with ultrasound was almost as good as the group treated with 50 mg/kg body weight of Bevacizumab without ultrasound. The result was consistent with the penetration experiment set forth above showing that the ultrasound assisted more penetration of Bevacizumab and thereby provided better chance of survival.

### CONCLUSION

In conclusion, the use of ultrasound induced blood-brain barrier opening enhances CNS delivery of the antiangiogenic mAb, bevacizumab. The CNS bevacizumab concentration is enhanced by up to 57 fold (FIG. 2a), resulting in significant anti-angiogenic effect on tumor periphery, and more importantly, overcome the current vascular normalization challenge of bevacizumab in GBM therapy. Moreover, a more profound therapeutic efficacy improvement was found in this study that combined ultrasound to enhance bevacizumab delivery contributes to 135% of median survival improvement, when compared with the improvement of 48% in bevacizumab administration alone (Table 1). This study provides proof-of-principle for development of therapeutic strategies that incorporate ultrasound to enhance CNS delivery of monoclonal antibody (or other large molecules), and suggests the ultrasound/bevacizumab combination as a promising anti-GBM strategy.

What is claimed is:

1. A method for reducing an effective amount of Bevacizumab required for treating brain tumor, comprising the following steps:
administering Bevacizumab to a subject; wherein said Bevacizumab is of an amount of 0.011 to 11 mg/kg body weight;
administering an ultrasound-response medium to said subject; and
administering said subject with an ultrasound exposure of 0.1 to 2 MI.

2. The method of claim 1, wherein said ultrasound exposure is of 0.47 to 1.26 MI.

3. The method of claim 1, wherein said ultrasound exposure is of 0.55 to 0.84 MI.

4. The method of claim 1, wherein said Bevacizumab is of an amount of 0.11 to 11 mg/kg body weight.

5. The method of claim 4, wherein said Bevacizumab is of an amount of 1.1 to 11 mg/kg body weight.

6. The method of claim 1, wherein said administering for said Bevacizumab is via intravenous injection.

7. The method of claim 1, wherein said administering for said ultrasound-response medium is via intravenous injection.

8. The method of claim 1, wherein said ultrasound-response medium is a plurality of particles.

9. The method of claim 8, wherein said particles have a mean diameter of 0.1 to 10 μm.

10. The method of claim 8, wherein said plurality of particles is of an amount of 1.9×10⁶ to 1.17×10⁸ particles/kg body weight for said administering.

11. The method of claim 8, wherein said particles are microbubbles.

12. The method of claim 1, wherein said ultrasound exposure is administered on the central nervous system of said subject.

13. The method of claim 1, wherein said brain tumor is Glioblastoma multiforme.

14. A kit for treating brain tumor, comprising:
a Bevacizumab formulation;
an ultrasound-response medium; and
an ultrasound system comprising an ultrasound transducer.

15. The kit of claim 14, wherein said Bevacizumab formulation comprises 0.11 to 25 mg/ml of Bevacizumab and a carrier for injection; wherein said mg/ml is based on the total volume of said Bevacizumab formulation.

16. The kit of claim 15, wherein said carrier for injection is water, saline, polymer, emulsifier, surfactant or a combination thereof.

17. The kit of claim 14, wherein said ultrasound-response medium comprises 1×10⁴ to 1×10¹² particle/ml of particles; wherein said particle/ml is based on the total volume of said ultrasound-response medium.

18. The kit of claim 17, wherein said particles have a mean diameter of 0.1 to 10 μm.

19. The kit of claim 14, wherein said ultrasound-response medium is mixed with a carrier for injection.

20. The kit of claim 19, wherein said carrier for injection is water, saline, polymer, emulsifier, surfactant or a combination thereof.

21. The kit of claim 14, wherein said brain tumor is Glioblastoma multiforme.

22. The kit of claim 14, wherein said ultrasound system is a medical imaging based guidance ultrasound system.

23. The kit of claim 22, wherein said medical imaging comprises neuronavigation, ultrasonography, optical imaging, computed tomography (CT), nuclear imaging, or magnetic resonance imaging (MRI).
